(19) United States
(12) Patent Application Publication
(10) Pub. No.: US 2011/0295309 A1
(43) Pub. Date: Dec. 1, 2011

(54) Combination belt buckle and tourniquet

(75) Inventors: Kelly Allen Sullivan, Alexandria, VA (US); Thomas Winfield Sullivan, Berryville, VA (US)

(73) Assignee: Kelly Allen Sullivan

(21) Appl. No.: 12/802,007

(22) Filed: May 27, 2010

(51) Int. Cl. A61B 17/00; A44B 11/02

(52) U.S. Cl. 606/203; 24/163 R

(57) ABSTRACT

A combination belt buckle and tourniquet includes a main body, a cover and a cover axle on which the main body and the cover are mounted so as to be rotatable relative to each other. When the combination is used as a tourniquet, webbing is fed between the main body and the cover, and the main body and the cover are rotated toward each other so as to form a pinching vice for capturing the webbing tightly therebetween.

### TECHNICAL FIELD

The invention relates to a combination belt buckle and tourniquet, and more particularly to a belt buckle which can be used as a tourniquet when a medical condition requires it.

### RELATED ART

Situations arise wherein medical personnel or other people encounter an injury such that a tourniquet is required to reduce or stop bleeding. In such a situation, a tourniquet may not be available, or at least the medical personnel or other persons must go to a medical vehicle, a medical storeroom, or other location to obtain the tourniquet, thereby delaying treatment.

Therefore, it would be advantageous if the medical personnel or other persons could have a tourniquet immediately available. For example, it would be advantageous if the medical personnel or other persons wore a belt buckle which could be converted into a tourniquet in such a situation as described above.

There have been schemes employed wherein a tourniquet has been implemented within a belt webbing, but such devices are not cleanly compact nor, in some cases, effective.

A tourniquet including a belt and buckle is disclosed in U.S. Pat. No. 4,640,281 of Sturm et al. However, there are certain deficiencies and/or disadvantages associated with the device of Sturm et al. For example, Sturm et al does not embody a means for grasping webbing or any lever means for forcefully applying mechanical tourniquet constriction. Although the device of Sturm et al is said to “clamp”, it does not have a “brake” because the device of Sturm et al does not have systemic means for tightening the tourniquet. Finally, the device of Sturm et al is physically configured in two separate or independent parts so that it provides two separate clamping configurations: one for the belt buckle application and the other for a tourniquet-clamp.

Thus, it would be desirable to have a belt buckle/tourniquet which is not hindered by the above disadvantages.

### DISCLOSURE OF INVENTION

The present invention relates to a combination belt buckle and tourniquet, and more particularly to a belt buckle which can be used as a tourniquet when a medical condition requires it.

Preferably, such a combination belt buckle and tourniquet comprises a main body, a cover and a cover axle on which the main body and the cover are mounted so as to be rotatable relative to each other wherein, when the combination is used as a tourniquet, webbing is fed between the main body and the cover, and the main body and the cover are rotated toward each other so as to form a pinching vice for capturing the webbing tightly therebetween.

As a further preference, such a combination belt buckle and tourniquet comprises a tourniquet lever having a fulcrum mounted thereon, and a pinching vice arm connected to said tourniquet lever via the fulcrum so as to be rotatable relative to the pinching vice arm, wherein an end of a webbing is fixed to a lower end of the tourniquet lever, is fed around a human body appendage, is fed between the tourniquet lever and the pinching vice arm, and is passed through an opening in the pinching vice arm, and wherein the pinching vice arm is rotated toward the tourniquet lever so as to capture the webbing between the tourniquet lever and the pinching vice arm.

It is an object of the present invention to provide a webbing belt and buckle assembly which can quickly convert into a tourniquet and, with little effort, be capable of applying sufficient force of constriction to the largest girth of a severely injured appendage.

It is a further object of the present invention to provide a combination belt buckle and tourniquet wherein the buckle is the complete mechanism for carrying out the required medical task, and wherein the belt assembly, when worn, presents a profile no larger than buckles presently used, is simplistic and rugged so as to endure the rigors of field service, and is not obtrusive when worn as a belt and buckle.

### BRIEF DESCRIPTION OF DRAWINGS

FIG. 1 is a perspective view of the device of the present invention employed as a belt buckle.

FIG. 2 is a perspective view of the various components or parts of the combination belt buckle and tourniquet of the present invention.

FIG. 3 is a side view of the device of the present invention with webbing threaded so as to form the tourniquet.

FIG. 4 is an isometric view of the device of the present invention employed as a belt buckle.

FIG. 5 is a frontal view of the device of the present invention with dimensions.

FIG. 6 is an isometric view of the belt buckle employed as a tourniquet in accordance with the invention.

FIG. 7 is a profile of the belt buckle with dimensions.

FIG. 8 is a side view of the device of the present invention with webbing threaded so as to form the belt with buckle.

### DETAILED DESCRIPTION

The invention will now be described in more detail with reference to the various figures of the drawings.

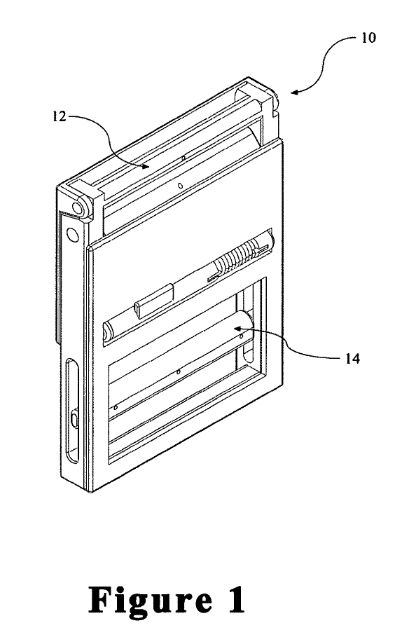

FIG. 1 is a perspective view of the device of the present invention employed as a belt buckle.

This perspective view is a view which would be exposed and seen when the belt buckle/tourniquet is worn. As seen in FIG. 1, the belt buckle/tourniquet 10 includes a belt fixed end bar 12 and a belt cinching jam 14.

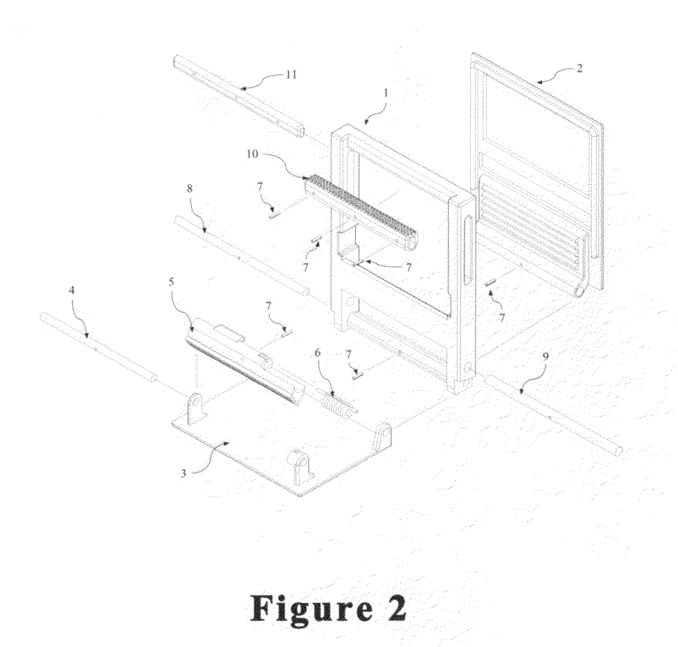

FIG. 2 is a perspective view of the various components or parts of the combination belt buckle and tourniquet of the present invention.

As seen in FIG. 2, the belt buckle/tourniquet of the present invention comprises a main body 1, a cover 2, a guard plate 3, a braking axle 4, a webbing brake 5, a return spring 6, coiled spring pins 7, a main body axle 8, a cover axle 9, a belt cinching jam 10, and a belt cinching jam slider 11.

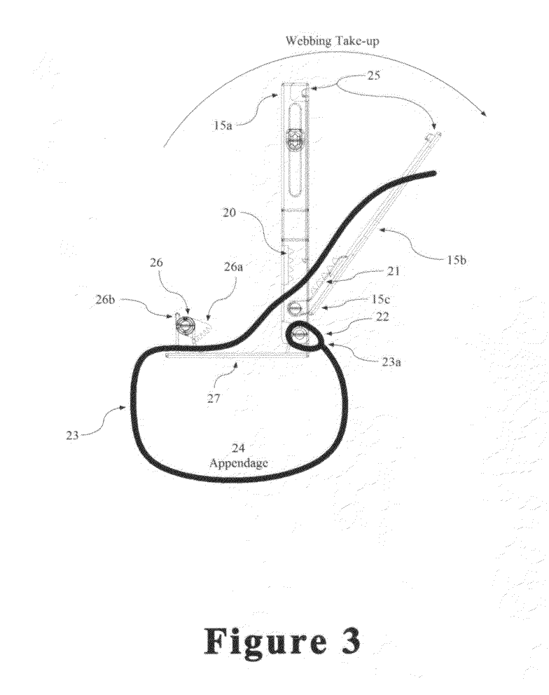

The hidden side of the main body 1 is provided with cleats which form a pinching vice (not shown in FIG. 2 but shown in FIG. 3 and discussed below), and which are used to capture the webbing between the main body 1 and the cover 2. All other components fold into and/or upon the main body 1.

The cover 2 is also cleated in complementary fashion relative to the main body 1. When the assembly is used as a tourniquet, webbing is fed between the main body 1 and the cover 2 and, by pinching a vice together, the webbing is captured tightly. The webbing thus captured can be drawn up by rotating the pinched vice on the axle 8 of the main body 1.

Guard plate 3 makes contact with the human body when applied as a tourniquet. This guard plate 3 also supports a brake 5. The guard plate 3 also provides a clean dimensional interface between the webbing, guard plate 3 and the human body, and avoids epidermal extrusion under the webbing during the tightening of the tourniquet.

Webbing fed under brake 5, that is, between the brake 5 and the guard plate 3, will move freely toward the main body 1. However, the braking action carried out by the brake 5 will prevent the webbing from moving in the opposite direction unless manually released. This is a one-way action used to retain the force on the webbing during tightening.

A return spring 6 is used to maintain contact between the brake 5 and the webbing 13. This facilitates and maximizes braking action when a take-up torque is relaxed.

A belt cinching jam 10 is used to tighten and retain the looped free end of a belt when the device is worn as a belt buckle. A belt cinching jam slider 11 fixes the assembly in the main body 1, while belt cinching jam 10 is surfaced to capture and retain the belt webbing when the assembly is worn as a belt buckle.

Coiled spring pins 7 are used to retain the assembled parts, while a cover axle 9 is an element about which the cover 2 pivots, and main body axle 8 is an element about which the main body 1 pivots. Brake axle 4 is an element about which the brake 5 pivots.

FIG. 3 is a side view of the device of the present invention with webbing threaded so as to form the tourniquet. This drawing demonstrates the mechanical operation of the belt buckle/tourniquet in the “tourniquet” mode.

The following elements are seen in FIG. 3: tourniquet lever 15a and pinching vice arm 15b which form a pinching vice 25, and which are connected by a fulcrum 15c about which the pinching vice arm 15b rotates; cleats 20 and 21 formed on the tourniquet lever 15a and the pinching vice arm 15b, respectively; lever fulcrum 22 about which the tourniquet lever 15a and the pinching vice arm 15b rotate together; webbing 23 having a fixed end 23a; appendage 24 of the person being treated; brake 26 (which includes brake cam 26a and tang 26b); and epidermal plate 27 connected to lever fulcrum 22.

Webbing end 23a is permanently fixed upon the buckle at lever fulcrum 22 as shown in FIG. 3. The webbing 23 is wrapped around the appendage 24, is fed under the brake 26, is threaded between the tourniquet lever arm 15a and the pinching vice arm 15b, and is finally threaded through an opening on the pinching vice arm 15b. With the pinching vice 25 relaxed, the tourniquet lever arm 15a is rotated backward (to the left) and then, using the thumb and forefinger, the pinching vice 25 is pinched together, thereby capturing the webbing 23 between the cleats 20 and 21 on the tourniquet lever arm 15a and the pinching vice arm 15b, respectively. With the pinching vice 25 pinched, holding the webbing 23 fast, the tourniquet lever arm 15a and the pinching vice arm 15b are rotated to the right about the lever fulcrum 22, drawing the webbing 23 tight. This process is repeated until the desired pressure upon the appendage 24 is achieved.

When the webbing 23 is relaxed from the pinching vice 25, the brake cam 26a with tang 26b captures and maintains tension upon the webbing 23. The length of the main body 1, the capture point of the webbing 23 and the position of the main body axle 8 (FIG. 2) yield approximately an 8:1 advantage when exerting force upon the webbing 23. Full relaxation of the tourniquet is accomplished by manually releasing the brake 26 using leverage developed at the tang 26b mounted on the brake cam 26a.

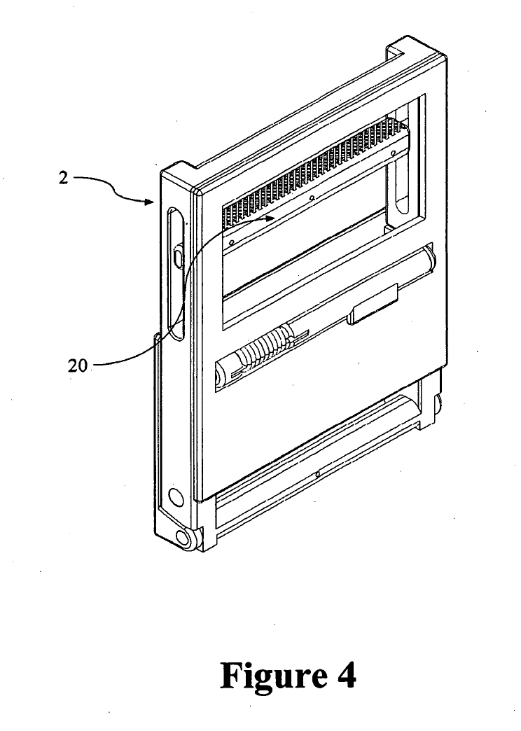

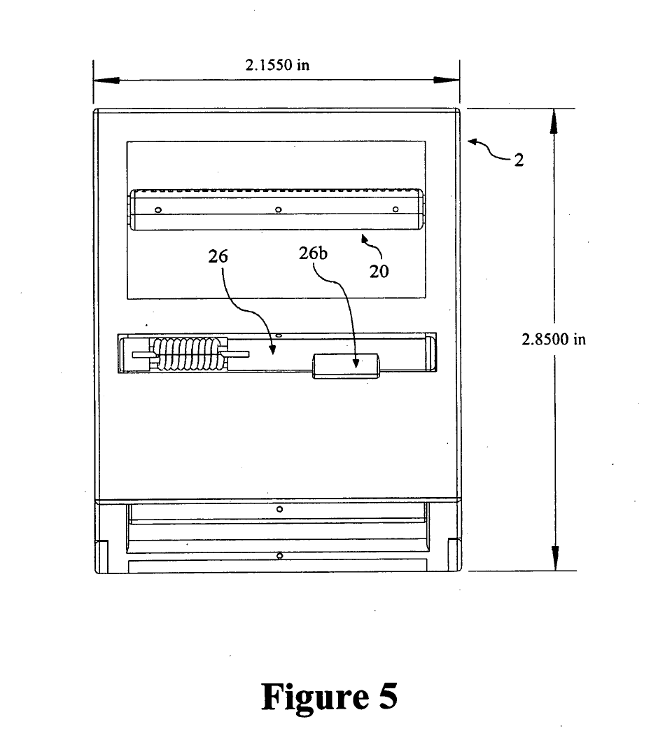

FIG. 4 is an isometric view of the device of the present invention employed as a belt buckle, while FIG. 5 is a frontal view of the device of the present invention with dimensions.

As seen in FIGS. 4 and 5, it is noted that the brake assembly slightly protrudes through the cover 2. Overlapping the cover 2 is the tang 26b of the brake 26. This is the method used to hold the full assembly in the closed position when employed as a belt buckle. Serving a dual purpose, this tang 26b is also used to manually manipulate the brake 26 when the assembly is employed as a tourniquet.

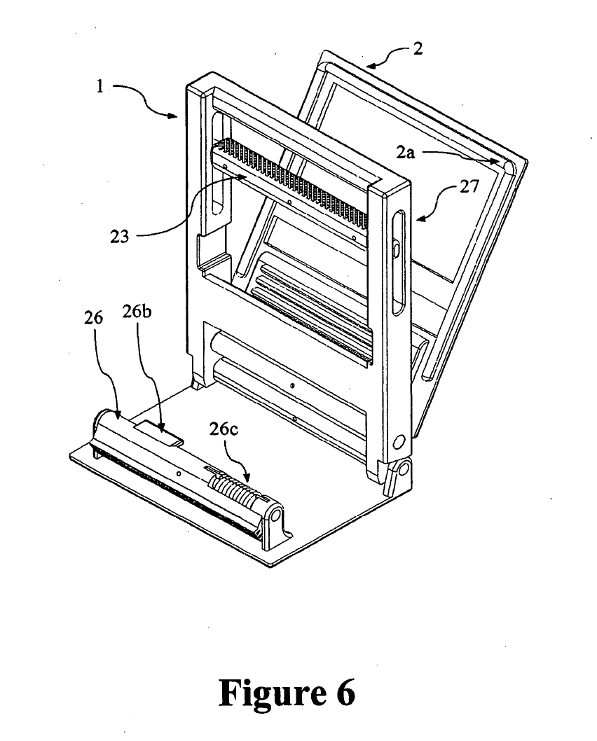

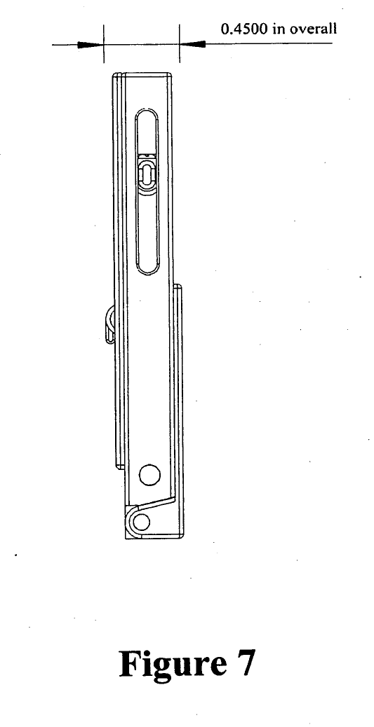

FIG. 6 is an isometric view of the belt buckle employed as a tourniquet in accordance with the invention, while FIG. 7 is a profile of the belt buckle with dimensions.

Referring to FIG. 6, the brake 26 with tang 26b and return spring 26c are shown. Also, a raised stiffener 2a traverses around the periphery inside the cover 2. This can be called a strong-back because it serves to strengthen this portion of the cover 2 so that it can endure the force required to pinch the webbing 23 tight within the pinching vice 25 (FIG. 3). The main body 1 has a conjugate recessed channel 27 which accommodates strengthening member 2a when the assembly is closed. Another aspect of this conjugate pair is that the cover 2 closes fast and secure against brutal field service.

Preferably, the material of the webbing 23 is woven nylon. This is a standard material used for service belts. The cleats 20 and 21 of the buckle are dimensioned so as to take advantage of the weave of webbing 23 when grasping.

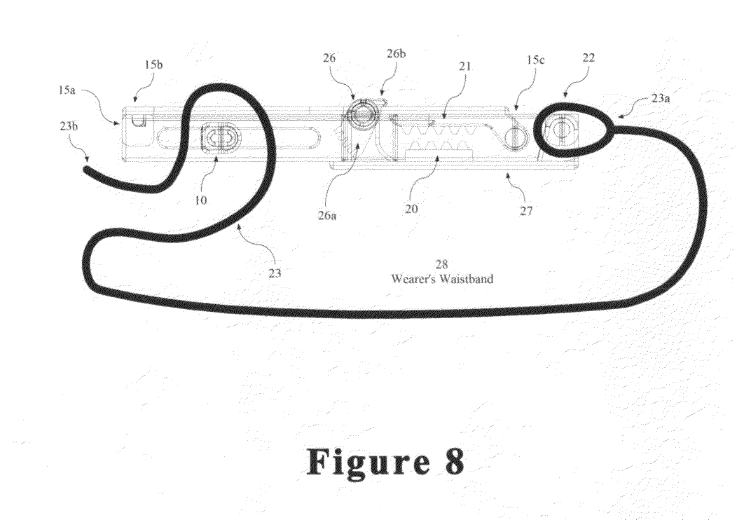

FIG. 8 is a side view of the device of the present invention with webbing threaded so as to form the belt with buckle. This drawing demonstrates the mechanical operation of the belt buckle/tourniquet in the “buckle” mode.

Converting from the tourniquet mode (FIG. 3) to the buckle mode, the device of the present invention as shown in FIG. 8 articulates as follows. Lever 15b pivots upon fulcrum 15c to lay flat upon lever 15a causing cleat 21 to store unseen in a space within lever 15a. Epidermal plate 27 pivots upon fulcrum 22 so as to lay flat upon lever 15a, causing the brake 26 with tang 26b to slightly protrude through an opening on lever 15b while brake cam 26a is stored unseen in a space within lever 15a. Finally, a preloaded force of return spring 6 (FIG. 2) causes brake 26 to rotate clockwise until tang 26b comes into contact with lever 15b, thereby latching the assembly into the buckle mode.

Webbing end 23a is permanently fixed upon the buckle at lever fulcrum 22 as shown in FIG. 8. The webbing end 23b is wrapped around the wearer's waistband 28, is threaded through openings of levers 15a and 15b to the right of cinching jam 10, is return treaded through openings of levers 15b and 15a to the left of cinching jam 10, and finally is manually pulled to the left, taking up slack in webbing 23 and causing cinching jam 10 to slide leftward, capturing webbing 23 between the cinching jam 10 and the inside surface of lever 15a.

While preferred forms and embodiments of the invention have been disclosed, it should be noted that various modifications known to those of ordinary skill in the art can be made without departing from the scope of the invention as defined in the appended claims.

1. A combination belt buckle and tourniquet, comprising:
a main body;
a cover; and
a cover axle on which the main body and the cover are mounted so as to be rotatable relative to each other;
wherein, when the combination is used as a tourniquet, webbing is fed between the main body and the cover, and the main body and the cover are rotated toward each other so as to form a pinching vice for capturing the webbing tightly therebetween.

2. The combination belt buckle and tourniquet of claim 1, further comprising a guard plate connected to said main body by means of a main body axle, and a brake mounted on the guard plate so that webbing, fed between the guard plate and the brake, moves freely toward the main body but is prevented from moving in an opposite direction unless braking action of the brake is released, thereby providing a one-way action used to retain force on the webbing.

3. The combination belt buckle and tourniquet of claim 2, further comprising a return spring used to maintain contact between the brake and the webbing.

4. The combination belt buckle and tourniquet of claim 2, wherein the guard plate provides a clean dimensioned interface between the webbing, the guard plate and a human appendage, and prevents epidermal extrusion under the webbing during tightening of the tourniquet.

5. The combination belt buckle and tourniquet of claim 1, further comprising a belt cinching jam which is surfaced to capture and retain webbing of a belt when the combination belt buckle and tourniquet is worn as a belt buckle.

6. The combination belt buckle and tourniquet of claim 1, further comprising a raised stiffener which traverses around a periphery inside the cover so as to strengthen the cover so that the cover can endure a force required to pinch the webbing tight between the main body and the cover.

7. A combination belt buckle and tourniquet, comprising:
a tourniquet lever having a fulcrum mounted thereon; and
a pinching vice arm connected to said tourniquet lever via the fulcrum so as to be rotatable relative to the tourniquet lever;
wherein an end of a webbing is fixed to a lower end of the tourniquet lever, is fed around a human body appendage, is fed between the tourniquet lever and the pinching vice arm, and is passed through an opening in the pinching vice arm; and
wherein the pinching vice arm is rotated toward the tourniquet lever so as to capture the webbing between the tourniquet lever and the pinching vice arm.

8. The combination belt buckle and tourniquet of claim 7, further comprising a lever fulcrum on which the tourniquet lever and the pinching vice arm are mounted so as to be rotatable together, wherein the tourniquet lever and the pinching vice arm are rotated together about the lever fulcrum so as to draw the webbing tighter until a desired pressure upon the human body appendage is achieved.

9. The combination belt buckle and tourniquet of claim 7, further comprising an epidermal plate connected to a lower end of the tourniquet lever, and having a brake mounted thereon for capturing the webbing and maintaining tension thereon.

10. The combination belt buckle and tourniquet of claim 9, wherein the brake comprises a brake cam and a tang.

11. The combination belt buckle and tourniquet of claim 7, further comprising a first cleat formed on the tourniquet lever and a second cleat formed on the pinching vice arm, wherein the webbing is captured between the first and second cleats on the tourniquet lever and the pinching vice arm, respectively, when the pinching vice arm is rotated toward the tourniquet lever.

12. The combination belt buckle and tourniquet of claim 7, further comprising a raised stiffener which traverses around a periphery inside the pinching vice arm so as to strengthen the pinching vice arm so that the pinching vice arm can endure a force required to pinch the webbing tight between the tourniquet lever and the pinching vice arm.
